(19) United States
(12) Patent Application Publication
(10) Pub. No.: US 2021/0393541 A1
(43) Pub. Date: Dec. 23, 2021

(54) PREPARATION OF NANOPARTICLES USING MODIFIED ICE-TEMPLATE

(71) Applicant: CITY UNIVERSITY OF HONG KONG, Kowloon (HK)

(72) Inventors: Chun-Sing LEE, Kowloon (HK); Shengliang LI, Kowloon (HK); Yingpeng WAN, Kowloon (HK); Zhongming HUANG, Kowloon (HK); Ming-Fai LO, Kowloon (HK)

(21) Appl. No.: 17/352,245

(22) Filed: Jun. 18, 2021

Related U.S. Application Data
(60) Provisional application No. 63/041,180, filed on Jun. 19, 2020.

(51) Int. Cl. A61K 9/51; A61K 31/12

(52) U.S. Cl. CPC A61K 9/5192; B82Y 5/00; A61K 31/12

(57) ABSTRACT

An improved ice-template method for the production of pure nanodrugs is disclosed, the method including application of a volume of a solution of the drug in 150 uL to a surface area of the ice template of about 200 mm².

### CROSS REFERENCE TO RELATED APPLIATIONS

This application claims priority to U.S. Provisional Application No. 63/041,180 filed with the United States Patent and Trademark Office on Jun. 19, 2020 and entitled “PREPARATION OF NANOPARTICLES USING MODIFIED ICE-TEMPLATE,” which is incorporated herein by reference in its entirety for all purposes.

The present invention relates to production of nanodrugs. In particular, the present invention relates to the production of nanodrug using an ice template.

### BACKGROUND

Nanodrugs are of great importance in the biomedical field due to several offered merits such as enhanced water dispersibility, bioavailability and improved tumour passive targeting ability.

Nanodrugs are particles of a drug which are so small that they are measured in the range of nanometres, typically in tens of nanometres. Due to their small size, nanodrugs can be finely dispersed in solvents in which the drugs are not naturally soluble. This provides a new mode of delivery and opens up the possibility of new and exciting properties that were not seen typically in the same drugs.

Conventionally, nanodrugs are made by re-precipitation, which exploits the difference in solubility of a drug in two miscible solvents, such as water and an organic solvent. Typically, the drug compound is first dissolved in the organic solvent, and the organic solution is dropped into water. Molecules of the drug will aggregate and precipitate as tiny nanoparticles in the water. However, several obvious weaknesses in re-precipitation method restrict its wide application, such as very low production rate and large batch-to-batch differences.

To improve production rate or nanodrugs, it has been proposed to precipitate hydrophobic drugs inside tiny pores formed in ice. This is generally known as the ice-template method. An ice template is ice formed of deionized water at minus 20 degrees Celsius. The method involves dripping multiple drops of a hydrophobic solution of a drug onto the surface of such a piece of ice, and letting the solution infuse tiny pores in the ice. Subsequently, ventilation is provided to the surface of the ice to evaporate the solvent, leaving behind the hydrophobic solute to precipitate in situ the pores. The tiny size of the pores restricts the solute from precipitating into particles any larger, thereby limiting the particle size of the resultant nanodrug. Subsequently, the ice is liquefied by melting and the nanoparticles collected by filtration.

At the current state of the art, the size of nanodrug particles obtained by the ice-template method is not uniform enough. The particle size spans too broadly a range of diameters. It is possible that this is because drops of the organic solution applied all over the surface of the ice template tend to flow and superpose as layers one over another. This creates inconsistent infusion of the solution into different parts of the ice template. Some of the drug precipitates inside pores of the ice to form smaller particles, but some of the drug precipitates on the ice surface, outside the pores, to form bigger particles.

Thus, there is a highly desirable need to propose methods or devices that can improve the ice-template method, so as to provide a possibility of producing nanodrugs that have particle size variations within a narrower range, i.e. better particle size uniformity.

### STATEMENT OF THE INVENTION

In a first aspect, the invention proposes a method of preparing nanoparticles of a pharmaceutical compound comprising the steps of: applying a hydrophobic solution containing the pharmaceutical compound onto a surface of ice, the surface confined by walls around the surface; the confined surface having area of about 200 mm²; the ice having pores; applying a volume of 50 μl to 150 μl of the solution onto the ice; the concentration of the solution being 1 mg/ml to about 50 mg/ml; ventilating the surface of the ice to remove the solvent and to precipitate the compound inside the pores of the ice.

Pores means holes in the ice and include capillaries.

The pores provide a space for precipitating particles of the drug. The size of the pores is typically in the nanometre range, possibly 150 nanometres or less, and preferably 50 nanometres or less. When the drug precipitates inside the pores, the size and width of the pores provide a spatial restriction which prevents formation of particles of the drug bigger than, typically, the width of the pores.

The method typically ends with removing the precipitate from the ice.

Preferably, the method comprises the further step of forming the ice in at least one well of a microplate, the diameter of the well being about 16 mm, or less. This provides that ice formed in the well has a surface that has the same diameter of 16 mm, or less.

The method is particularly advantageous for producing nanoparticles of curcumin, in which case the solution is of curcumin dissolved in tetrahydrofuran at concentration of 1 mg/ml to 50 mg/ml. However, it is more preferable that the solution is of curcumin dissolved in tetrahydrofuran at concentration of 10 mg/ml, which possibly gives a particle size most suitable for medical use.

In a second aspect, the invention proposes a piece of ice; the piece of ice having wall defining surface for receiving a drug solution; the surface has an area that is substantially equivalent to an area defined by a diameter of 16 mm; the ice embedded with nanoparticles of a hydrophobic pharmaceutical compound; the nanoparticles formed in-situ inside pores in the ice.

Typically, the piece of ice is formed in a well in a 24-well microplate; the well providing the confined surface.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 shows a microplate used in an embodiment of the present invention;

FIG. 2a shows the plan view of the microplate of FIG. 1;

FIG. 2b shows how the microplate of FIG. 1 is used in the embodiment of the present invention;

FIG. 3 is a closed up view of one of the steps in FIG. 2b;

FIG. 4 illustrates a possible mechanism in the method of FIG. 2b;

FIG. 5 shows another embodiment, alternative to that of FIG. 1;

FIG. 6 shows another embodiment, alternative to that of FIG. 1.

FIG. 7 is a closed up view of using the microplate of FIG. 5;

FIG. 8 shows a possible mechanism which results from using the microplate of FIG. 5 in the prior art;

FIG. 9 shows the molecule of curcumin, which is used in an example of how the embodiment of FIG. 2b is deployed;

FIG. 10, FIG. 11, FIG. 12, FIG. 13 and FIG. 14 shows the results of using the microplate of FIG. 1 over using the microplate of FIG. 5; and

FIG. 15 shows the efficacy of a nanodrug obtained using the method of FIG. 2b.

### DESCRIPTION OF EMBODIMENTS

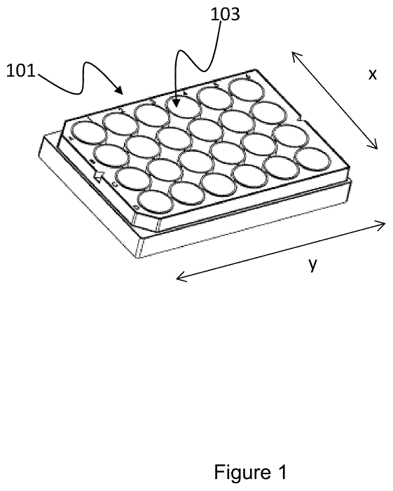

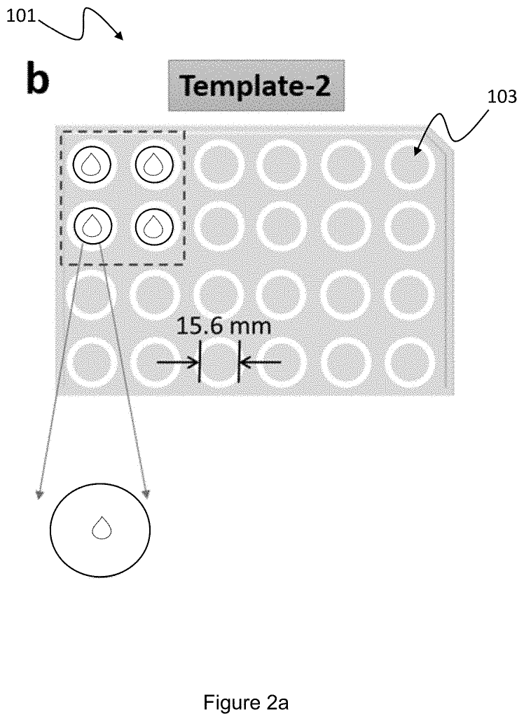

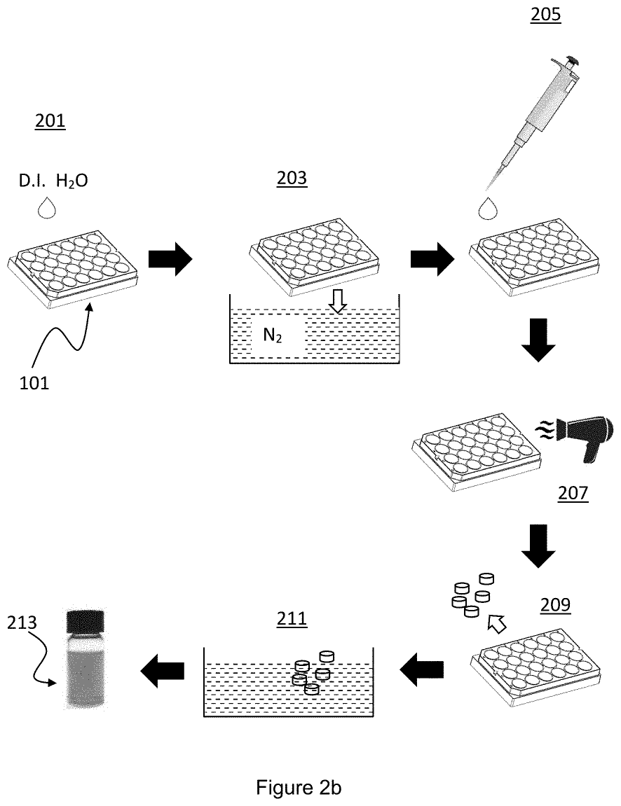

FIG. 1 shows a polysterene multi-well microplate 101, which is usually used for cell culture and has 24 circular wells 103 (image obtained from https://www.n-genetics.com/products/1236/1023/13099.pdf). The plan view of the microplate 101 is shown in FIG. 2a. Each well 103 has a top diameter and depth of 15.6 mm×10 mm (diameter×height). Therefore the top of the well 103 has an area of about 191.1 mm² (1.911 cm²). When water is provided into the wells 103 and frozen, ice forms in the well 103. The area of the top surface of the ice is defined by the size of the mouth of the well 103. In other words, the walls of each well 103 confine or limit the flow of any fluid placed onto the top surface of the ice within the well 103.

FIG. 2b shows how the microplate 101 is used to make an ice-template for producing pure nanodrug (PND) particles. “Ice-template” is used loosely in this description to described ice that is suitable for use in producing nanoparticles of a drug. At first, each of the wells 103 in the microplate 101 is filled with de-ionised water, at 201, and flash-frozen at minus 20 degrees Celsius to produce ice in the wells 103. The flash frozen ice provides the ice-template.

Typically, the microplate 101 is can be placed in a freezer at minus 20 degrees Celsius for 10 hours to flash freeze the deionised water. More preferably, however, the base of the microplate 101 can be immersed into a liquid nitrogen bath, at 203, to flash freeze the de-ionised water before transferring the microplate 101 into a freezer at minus 20 degrees Celsius for 10 hours to prepare the ice for infusion with the hydrophobic solution. As the skilled man knows, liquid nitrogen has a temperature of minus 196 degrees Celsius, making the freezing even quicker than in the freezer of minus 20 degrees Celsius.

Ice suitable for use as ice template has multiple tiny, nano-size pores or capillaries in the nano-size range. One possible reason for the formation of these tiny pores is the flash freezing at a temperature of minus 20 degrees Celsius or lower. As the skilled reader would know, flash freezing creates many points of nucleation in the deionised water resulting in the growth of many disconnected ice crystals, and the spaces between the ice crystals provide the pores or capillaries.

Flash freezing is provided by the huge temperature difference between the natural freezing point of zero degrees Celsius, and to some extent by the small volume of water in each well 103 that makes conduction of heat away from the water easier or speedier.

Therefore, it is proposed that, separating the deionized water into different, small portions according to the plurality of wells 103 in the 24-well microplate 101 allows faster chilling of the water to create multiple sites of nucleation, increasing the certainty of creating more similarly sized pores. However, the skilled reader would understand that use of relatively small wells to create ice is a preferred feature but not a requirement in every embodiment of the invention.

Before being applied onto the surface of the ice, the drug is first dissolved in a hydrophobic solvent with relatively low boiling point. A single shot of the ensuing hydrophobic solution is dropped onto the surface of each piece of the ice formed in the wells 103, at 205, using a pipet. To keep the ice from melting, the microplate 101 is kept on an ice pedestal (not illustrated) while the hydrophobic solution is being applied onto the top surface of the pieces of ice, and also while the solution is given time to infuse into pores in the ice.

A unique property of ice is that surface of the pores in the ice is lined with relatively mobile water molecules, which behaves like liquid. Hence, the pore surface assists infusion of the hydrophobic solution into the ice.

Optionally, the ice, with the solution dropped onto the top surface of the ice, is then placed in a freezer at minus 20 degrees Celsius for 10 hours to let the solution continue to infuse into the ice pores. Keeping the ice template at a very low temperature prevents enlargement and amalgamation of the ice crystals, sustaining the tiny pores in the ice.

Subsequently, the solvent is removed from the ice template by passing a flow of air or insert gas over the surface of the ice, at 207. This evaporates the solvent from the ice template. As the solvent leaves the ice template, the solute is forced to precipitate inside the pores in the ice.

Upon complete evaporation of the solvent, the drug-loaded ice is left to age for a further 24 hours at minus 20 degrees Celsius, during which molecules of the drug further self-assemble into nano-sized particles, i.e. pure nanodrug or PND.

Finally, the pieces of ice are removed from wells 103 of the 24-well microplate 101, at 209. The removed drug-filled pieces of ice are simply melted, at 211, in a bath of deionised water to yield a colloidal suspension of the nanodrug 213. Preferably, sonication is applied for 10 to 30 minutes as the ice melts to assist dispersion of the nanodrug 213 in the colloidal suspension. The nanodrug 213 can be recovered by a vacuum filtration system. Alternatively, instead of melting the ice, the nanodrug 213 is separated from the ice by freeze-drying (not illustrated) in vacuum, or sublimation, to yield a dry powder of the nanodrug 213 (not illustrated).

In some other embodiments, where it is plausible depending on the type of drug to be precipitated, the step of removing the solvent from the ice can be done in a mild vacuum over a period of time, followed by sublimation of the ice in a greater vacuum.

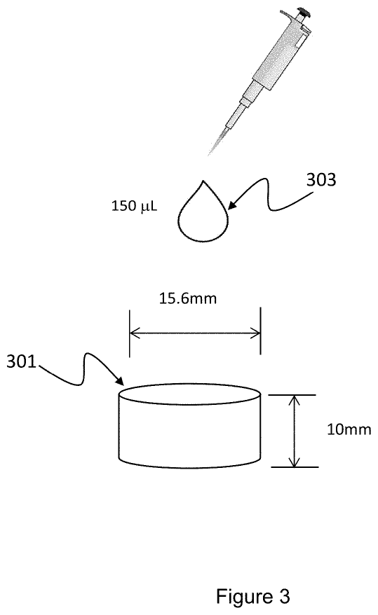

FIG. 3 shows the process of applying a drop of the solution 303 onto a piece of ice 301 in one of the wells 103 in the 24-well microplate 101. The wells and the microplate are invisible in FIG. 3 for clearer illustration. For such a surface area, the preferred drop size of the hydrophobic solution is 150 μl. This ratio of the surface area to volume of the solution is therefore about 191.1 mm² or less (1.911 cm²): 150 μl. That is, or 200 mm² or less: 150 μl. This ratio of surface area to the volume of the solution dropped onto the surface has been found to provide very small and uniformly sized nanoparticles of the drug. All the 24-wells in the microplate 101 can each be infused with the solution of this volume.

The depth of the ice 301 is less important regarding the infusion of the solution, but may be relevant relating to the flash freezing of the ice 301 to provide the ice template, as the speed of flash freezing is related to the volume of the water in the well 103.

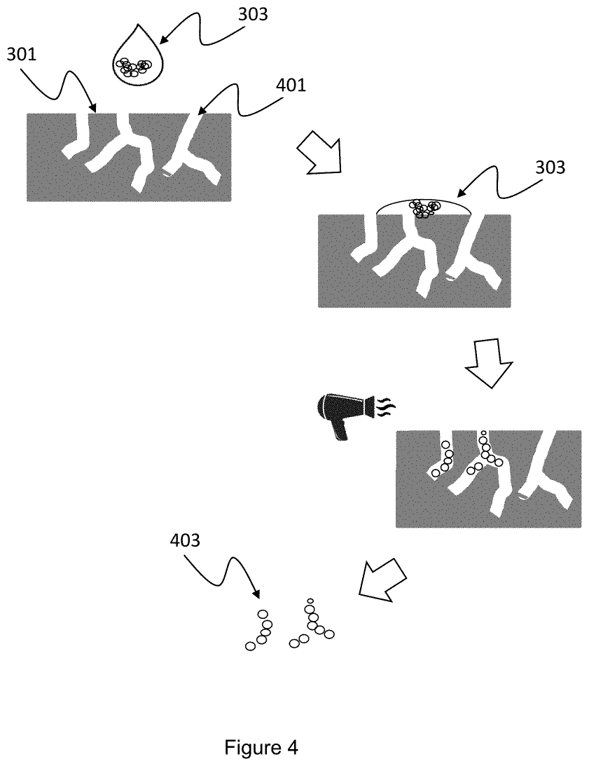

FIG. 4 further shows how the solution is able to infuse the pores in the ice 301. A suitable volume of the solution 303 the drug is dropped onto the surface of the ice 301. The solution can seep into the pores in the ice 301 slowly and eventually occupy the pores 401.

Given time, the solution 303 is able to infuse into the ice 301 fully. Preferably, however, further drops of pure solvent without the solute are applied to the surface of the ice 301, over the earlier applied hydrophobic solution containing the dissolved drug, to push traces of the solution further into the pores to increase the likelihood that all of the drug precipitates inside the pores.

Accordingly, the walls or boundaries of the wells 103 in the microplate 101 define a specific surface on the ice, upon which a drug solution may be introduced onto the ice 301. For an ice surface of given area, it is possible to calculate the optimal volume of the hydrophobic solution that may penetrate into the pores of the ice 301 fully, without over supplying the area with solution. This provides a possibility of preventing superposing layers of solution from applying multiple drops of the solution in an attempt to utilise an overly large area.

Finally, the solvent is removed from the ice 301 by ventilating the top surface of the ice 301. When the solvent has been evaporated away, the solute is left behind as precipitate 403 of the drug. The extent of amalgamation of the precipitate is restrained by the size of the pores 401 in the ice, which is in the nano-metre range. As mentioned, it is preferable that the nanoparticles are left to age in the ice 301 for a period of time before extracting the nanoparticles, such as for another 10 hours, which allows the nanoparticles to re-arrange themselves and stabilize inside the pores.

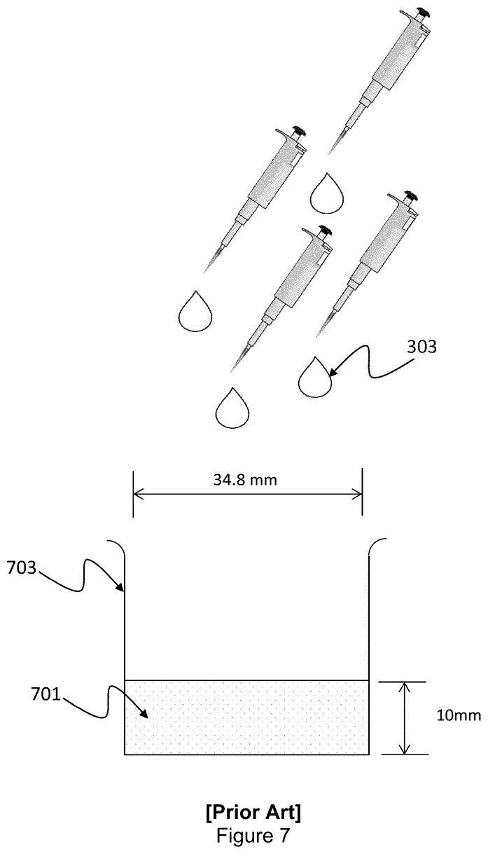

FIG. 7 illustrates a comparative prior art ice template, which is a slab of ice 701 formed in a small beaker 703. The slab of ice 701 can be of any shape but if made in a small beaker 703 is often round.

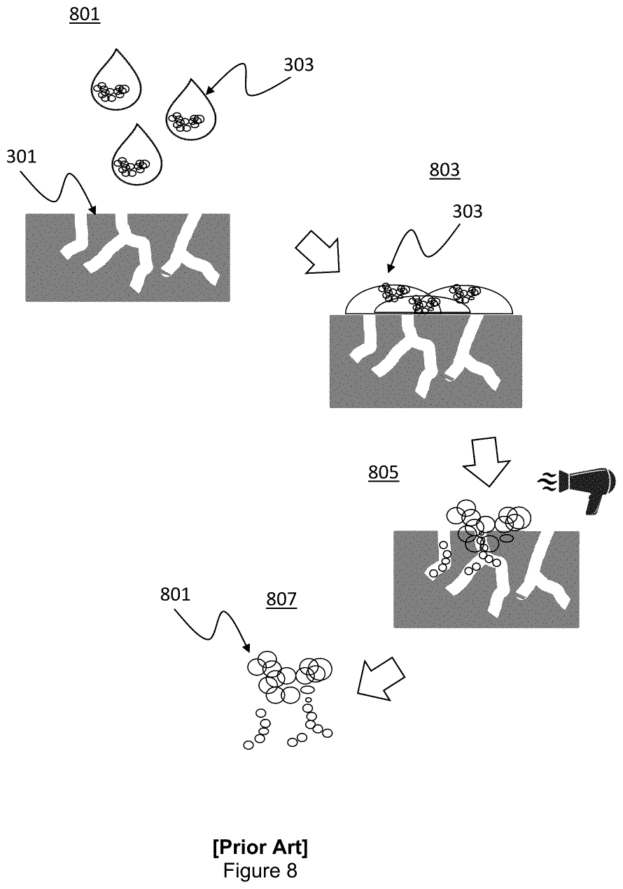

Just as it has been described for the 24-well microplate 101, to produce ice in a beaker 703, the beaker 703 is filled with deionized water and flash frozen for 10 hour of freezing at minus 20 degrees Celsius. The ice 701 in the beaker 703 is significantly larger than the ice 301 made in each well 103 of the 24-well microplate 101 of FIG. 1. Therefore, to fully use the ice 701 formed in the beaker 703 to make nanoparticles of the drug, the drug solution is dropped all over the surface area of the ice 701 as illustrated in FIG. 7 and in FIG. 8, at 801. However, it is difficult for the technician to drop the solution evenly over all areas of the ice 701, as the solution is prone to flowing. Therefore, the solution of one drop can flow and form a layer over another layer of the solution. Hence, as shown in FIG. 8, this can create localized areas of multiple layers of the solution, which may not all have seeped into the pores of the ice fully, at 803.

This possibly causes some of the drug to precipitate outside the restraint of the pore space, at 805, precipitating relatively large drug particles when the solvent is removed by ventilation, which expands the range of the particle size of the nanodrug 213, and reducing particle size uniformity of the nanodrug 213. As a result, nanodrugs produced this way have a broad range of particle size, at 807.

It is not desirable to apply a single drop of the solution onto the ice 701, as the overall size of the ice 701 takes up precious space in the freezer, and is therefore not economical or highly productive.

Comparing with prior art of FIG. 7 to FIG. 8, the walls defining the wells 103 in the microplate 101 of FIG. 1 to FIG. 3 provide multiple, functionally separated, confined, topside ice surfaces that are relatively small compared to a slab of ice produced using a beaker 703.

Accordingly, the embodiment provides a plurality of areas, each area suitable for infusion with a drop of the solution of the drug, without need of multiple drops of the solution to cover the entire surface. This allows the technician to apply a single drop of the solution into each well 103 relatively quickly, while preventing superposing of multiple drops of the solution in a single area. That is, 24 drops of the solution can be applied into respective well 103 in the microplate 101 of FIG. 1, each drop confined and segregated by the boundaries of the wells 103.

This provides further advantage that the technician need not be very highly skilled in applying the solution over the surface of the ice evenly, as the technician is assisted by the confinement around the top surface of each piece of the ice in the wells 103.

### EXAMPLE

Preparation of Curcumin Nanodrug

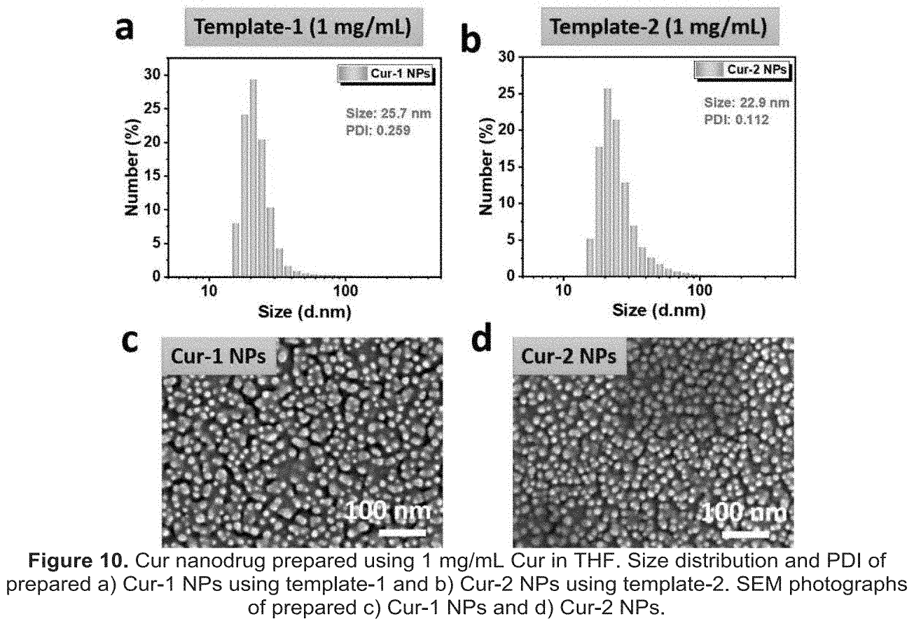

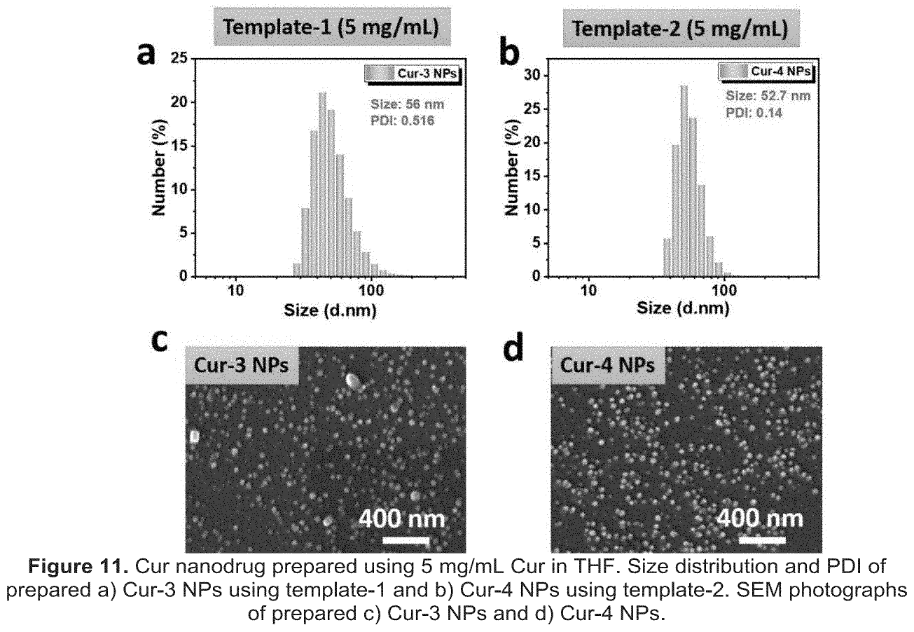

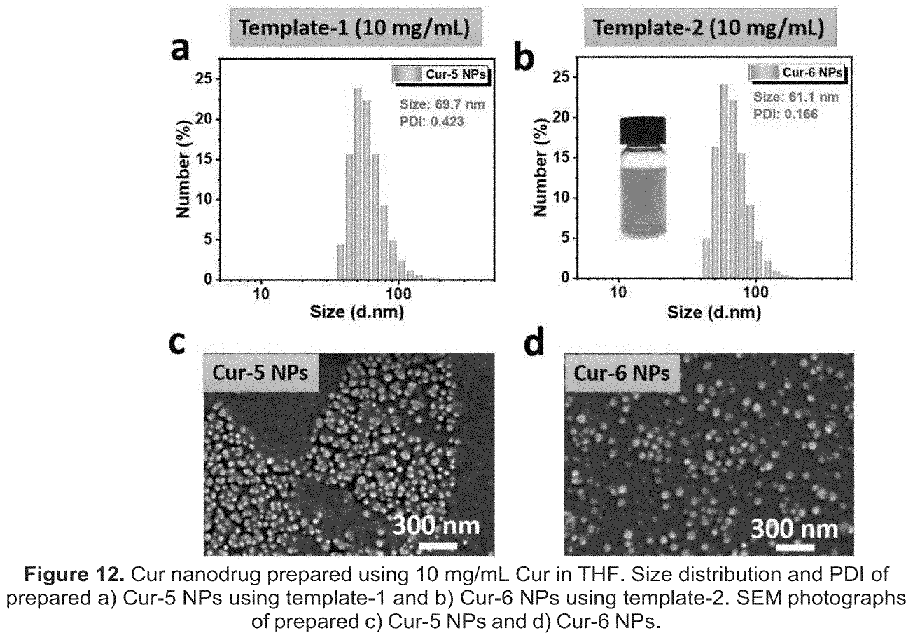

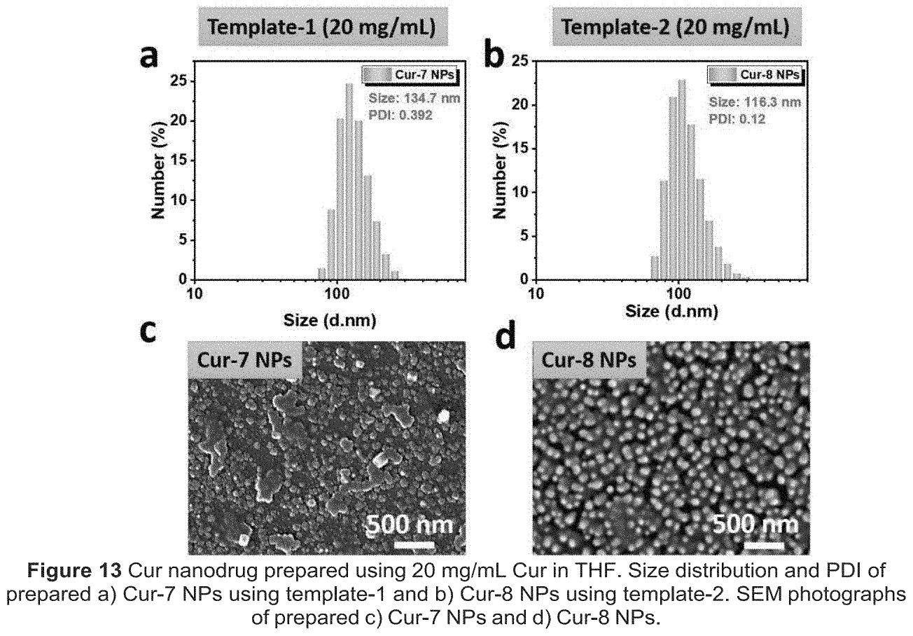

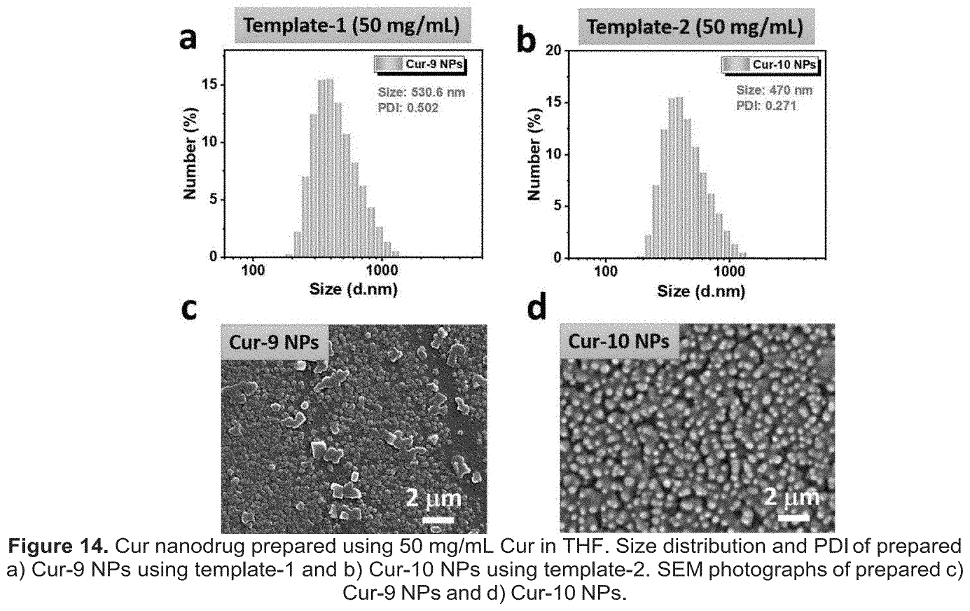

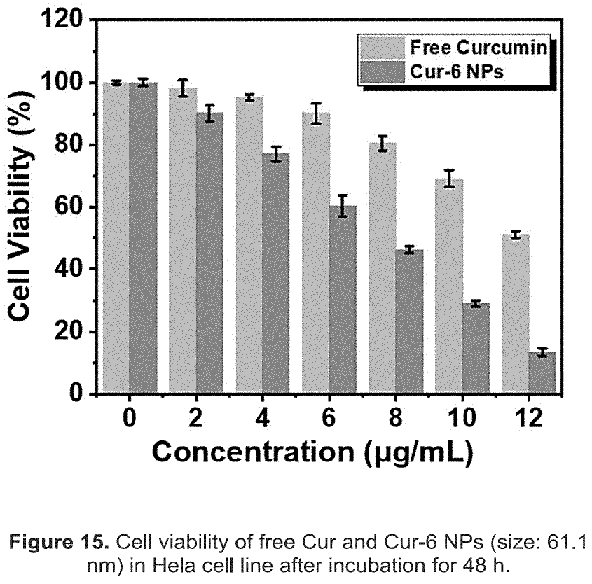

By way of example, the present method has been used to produce nanoparticles of hydrophobic drug molecule curcumin (Cur), and the superior performance of the present method is demonstrated in FIGS. 10 to 15.

In the example, beakers and a 24-well microplate 101 were both used to make pieces of ice of different sizes to be used as ice templates.

The beakers have a diameter of 34.8 mm, while the wells of the 24-well microplate 101 have a diameter of 15.6 mm, or about 16 mm. The beakers were each filled with 3 mL pure deionized water, while the 24-well microplate 101 was filled with 1 mL DI water per well, and stored in minus 20 degrees Celsius. The microplate 101 and the beakers were stored in minus 20 degrees Celsius for 10 hours to produce a plurality of ice templates.

Results from using the ice-templates made in beakers are labelled “template-1”. Results from using the ice-templates made using the 24-well microplate 101 are labelled “template-2”.

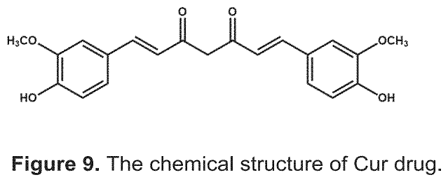

The chemical structure of curcumin molecule was shown in FIG. 9. Curcumin was dissolved in tetrahydrofuran (THF) to produce solutions of concentrations of 1, 5, 10, 20, 50 mg/ml. A pipet is used to apply 150 μl of these solutions of different concentrations onto the surface of the different ice templates. When applying the solution onto the ice templates, the ice templates were placed on an ice pedestal in order to avoid melting of ice templates.

Subsequently, the THF is removed from the ice templates by supplying air flow across the surface of the ice templates. Consequently, curcumin nanoparticles are formed inside the pores of the ice.

The ice templates, infused with the drug now, are put in a freezer of minus 20 degrees Celsius for another 12 hours to age the nanoparticles.

Eventually, the ice templates were left to melt separately in room temperature. Sonication is applied as the ice templates melt. Aqueous colloidal suspensions of the nanodrug were thereby obtained.

The particle size distribution of curcumin obtained by the difference ice templates are shown in FIGS. 10 to 14, along with SEM photographs shown the nanoparticles. A picture of a vial of nanodrug colloidal dispersion is shown as an inset in FIG. 12b.

FIG. 10 shows the polydispersity index (PDI) of the curcumin nanoparticles prepared by using a Cur-THF solution (curcumin dissolved in tetrahydrofuran) of 1 mg/ml. The particle size distribution of the nanodrug obtained in in the beaker is shown as FIG. 10a, i.e. template-1, and the corresponding SEM image is shown in FIG. 10c, i.e. Cur-1.

The particle size distribution of the nanodrug obtained in in the 24-well microplate 101 is shown as FIG. 10b, i.e. template-2, and the corresponding SEM image is shown in FIG. 10d, i.e. Cur-2.

It can be seen in FIG. 10b that the particle size of the Cur-2 nanodrug, obtained by using current new method, is significantly smaller than the Cur-1 nanoparticles made by the beaker ice template method seen in FIG. 10a. That is, the size of Cur-2 is smaller at 22.9 nm at a PDI of 0.112, while the size of Cur-1 is 25.7 nm on the average with a PDI of 0.259.

As may be seen in all the data and pictures shown in FIG. 10 to FIG. 14, the better PDI of prepared nanodrug were obtained in all groups using the modified template-2, i.e. the 24-well microplate 101.

Size uniformity is important in controlling the medical effects of nano drug. The optimal nanodrug has to avoid clearance by reticuloendothelial system (RES) and filtered by kidney finally achieving long circulation in body. Nanoparticles with uniform size between 50 nm and 100 nm are required for the best circulation results according to previous research. Therefore, the nanodrug labelled Cur-6 prepared using modified template-2, and shown in FIG. 12b, having a suitable particle size of 61.1 nm, was used for final anticancer application. FIG. 8 shows that the Cur-6 nanodrug has have better anticancer effect than free curcumin drug for Hela cell line.

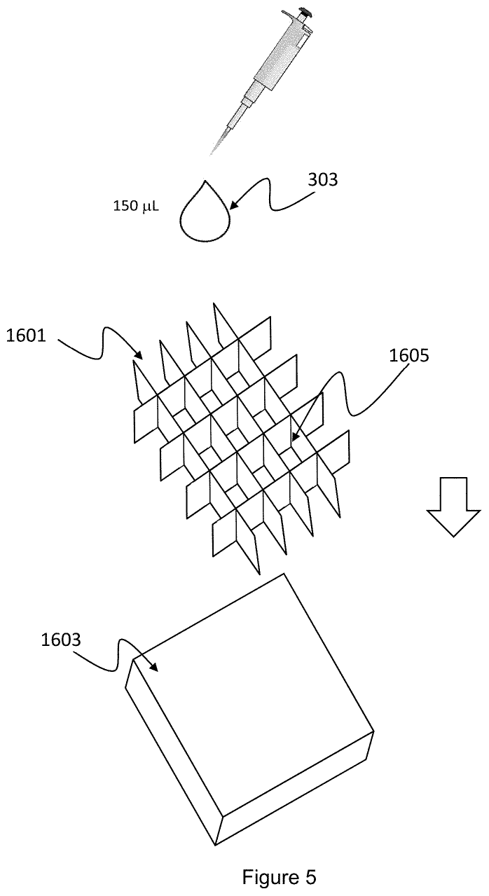

FIG. 5 shows another embodiment of the same invention, which does not use a microplate. In this embodiment, the ice is a singular, large piece of ice 1603. To provide confine and separate surfaces on the ice 1603 for infusion with the hydrophobic solution of a drug, a frame 1601 is provided for placing onto a surface of the large piece of ice.

The frame 1601 in FIG. 5 is an integral construction of a plurality of parallel plastic panels that are crossed orthogonally with another plurality of plastic panels, which define multiple square cells 1605, which has a fluid separation function like the wells 103 of the microplate 101 of FIG. 1. The frame 1601 can be made in any other configuration, although FIG. 5 only shows one example of a frame.

The dimensions of the frame 1601 are such that it is suitable for being placed on the surface of the piece of ice, and the square cells 1605 of the frame 1601 each provides confinement of an area on the surface of the ice. Each of these areas is suitable for being applied with a suitable amount of drug solution. Advantageously, the solution applied in each of the areas is unable to flow over to the neighbouring area, being separated by the frame 1601.

The skilled reader would understand that the dimensions and configurations of the plastic panels, and therefore the size of the areas defined by the frame, can be varied according to the type of drug, the solvent and the concentration of the solution to be applied onto the ice surface. Hence, it is not necessary to give specific dimensions and measurements here. It suffices to state that, if the frame 1601 is used to divide the ice slab for infusion with the afore-described curcumin solution, then each cell 1605 preferably has dimensions that define areas of about 200 mm² each on the surface of the ice slab 1603, such that a volume of 50 μl to 150 μl of the solution can be applied to onto each of the areas, where the concentration of the drug, such as curcumin, in the solution is 1 mg/ml to about 50 mg/ml.

After the drug solution has been infused into the ice, the frame 1601 can be removed before ventilation is applied to remove the solvent. As the surface of the ice slab 1603 without the frame 1601 has no obstruction to flow of air, this embodiment provides that it is easier to remove the solvent from the ice by ventilation.

An advantage of using the frame 1601 is that space is more economically used; the cells 1605 defined by the frame 1601 are not separated from each other by a distance, unlike the wells 103 in the microplate 101, as shown in FIG. 1. That is, the ells 1605 defined in the frame 1601 are even more compactly arranged that the wells 103 in the microplate 101.

Optionally, the frame 1601 can be submerged slightly into a tray of water, and remains in the water as the water is flash frozen. In this way, any part of the frame 1601 that is protruding from the surface of the ice acts like the well in the microplate embodiment of FIG. 1 (not illustrated).

Without intention to be restricted to any particular shape, the preferred area of the surface ice for receiving the solution is substantially or somewhat equivalent to that of a circular area having a diameter of 16 mm.

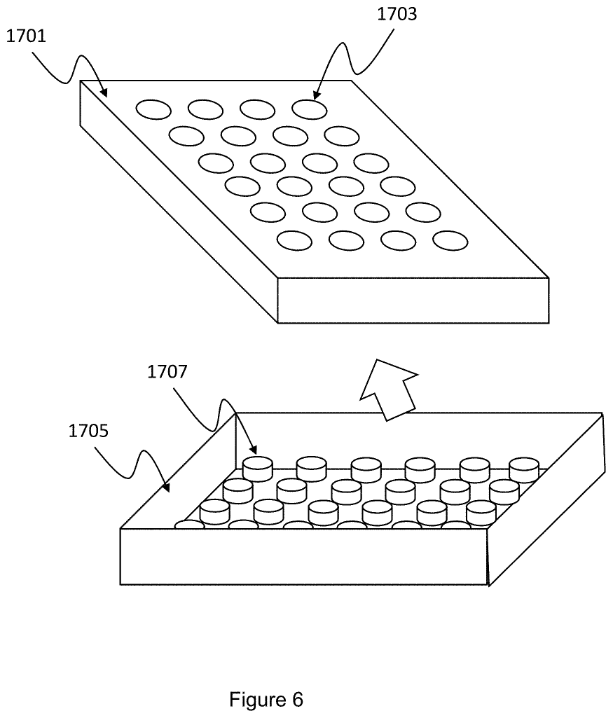

FIG. 6 shows yet a further embodiment. In this embodiment, no microplate or frame is used to define the separate areas of ice to be infused with drug solution. A mould 1705, like an ice tray, is provided to make the embodiment. The mould 1705 is filled with deionised water, and flash frozen to form a singular, large piece of ice 1701. The base of the mould 1705 has protrusions 1707 that create depressions 1703 or wells into the piece of ice. When the ice 1701 is removed from the mould 1705 and turned over, the top surface of the ice 1701 now has multiple depressions 1703, each suitable for being filled with one drop of a drug solution at an appropriate concentration. In particular, if the drug to be made into a nanodrug is curcumin as afore-described, each of the depressions preferably has a bottom surface area that is similar to the area of defined by the mouth of each well 103 in the 24-well microplate 101. That is, the base of each depression 1703 has an area of 200 mm², such that a volume of 50 μl to 150 μl of the curcumin solution can be applied into each depression 1703, where the concentration of the curcumin solution is 1 mg/ml to about 50 mg/ml. This embodiment has the added advantage that a foreign material in the form of a frame or a plastic tray does not have to come into contact with the solution. This avoids any chemical affinity, contamination or loss of yield from contact between hydrophobic solution and organic plastic.

Accordingly, the embodiments include a method of preparing nanoparticles (i.e. nanodrug 213) of a pharmaceutical compound (i.e. the drug) comprising the steps of: applying a hydrophobic solution containing the pharmaceutical compound onto a surface of ice (i.e. ice template), the surface confined by walls around the surface; the confined surface having area of about 200 mm²; the ice having pores; applying a volume of 50 μl to 150 μl of the solution onto the ice; the concentration of the solution being 1 mg/ml to about 50 mg/ml; ventilating the surface of the ice to remove the solvent and to precipitate the compound inside the pores of the ice.

The embodiments also include a piece of ice; the piece of ice having wall defining surface for receiving a drug solution; the surface has an area that is substantially equivalent to an area defined by a diameter of 16 mm; the ice embedded with nanoparticles of a hydrophobic pharmaceutical compound; the nanoparticles formed in-situ inside pores in the ice.

While there has been described in the foregoing description preferred embodiments of the present invention, it will be understood by those skilled in the technology concerned that many variations or modifications in details of design, construction or operation may be made without departing from the scope of the present invention as claimed.

For example, where it is described that solvent in the ice is removed by movements of air or inert gas, the skilled reader should appreciate that other methods of removing the solvent is within the contemplation of this application, such as by placing the ice in a relatively low pressure or mild vacuum environment to encourage vaporization of the solvent.

Although minus 20 degrees Celsius is mentioned, other lower temperatures are useable to flash freeze water into ice suitable for use as ice template to make nanoparticles of drugs.

Beside the surface area of the ice template, and depending on the type of drug, the concentration of the solution may have an effect on the final particle size. Generally, the spread of the size of the nanoparticles decreases as the concentration of the drug solution decreases. Furthermore, the temperature of the solution has an effect on the final particle size. Decreasing temperature of drug solution to 4 degrees Celsius before applying onto the ice template can substantially reduce the average particle size. The possible reason for this is that when a relatively warm temperature drug solution is loaded onto an ice sheet, the solution may melt the surface of its contacted ice grains slightly, which widen the pores, leading to large particle size growth inside the larger pores. All these factors are variables that may be optimised in actual production and need not be addressed herein.

1. A method of preparing nanoparticles of a pharmaceutical compound comprising the steps of:
applying a hydrophobic solution containing the pharmaceutical compound onto a surface of ice, the surface confined by walls around the surface;
the confined surface having area of about 200 mm²;
the ice having pores;
applying a volume of 50 μl to 150 μl of the solution onto the ice;
the concentration of the solution being 1 mg/ml to about 50 mg/ml;
ventilating the surface of the ice to remove the solvent and to precipitate the compound inside the pores of the ice;
removing the precipitate from the ice.

2. A method of preparing nanoparticles of a pharmaceutical compound as claimed in claim 1, further comprising the step of:
forming the ice in at least one well of a microplate, the diameter of the well being about 16 mm or less.

3. A method of preparing nanoparticles of a pharmaceutical compound as claimed in claim 1, further comprising the further step of:
applying solvent used in the solution to wash the solution deeper into the pores of the ice.

4. A method of preparing nanoparticles of a pharmaceutical compound as claimed in claim 2, further comprising the following steps for providing the ice:
filling the well with 1 ml of deionized water;
flash freezing the deionized water in the well.

5. A method of preparing nanoparticles of a pharmaceutical compound as claimed in claim 4, wherein the step of flash freezing the deionized water in the well comprises:
flash freezing deionized water in a freezer at minus 20 degrees Celsius.

6. A method of preparing nanoparticles of a pharmaceutical compound as claimed in claim 4, wherein the step of flash freezing the deionized water in the well comprises:
flash freezing deionized water in a bath of liquid nitrogen.

7. A method of preparing nanoparticles of a pharmaceutical compound as claimed in claim 1, wherein the pharmaceutical compound is curcumin; and
solution is of curcumin dissolved in tetrahydrofuran at concentration of 1 to 50 mg/ml.

8. A method of preparing nanoparticles of a pharmaceutical compound as claimed in claim 1, wherein
the pharmaceutical compound is curcumin; and
solution is of curcumin dissolved in tetrahydrofuran at concentration of 10 mg/ml.

9. A piece of ice;
the piece of ice having wall defining surface for receiving a drug solution;
the surface has an area that is substantially equivalent to an area defined by a diameter of 16 mm;
the ice embedded with nanoparticles of a hydrophobic pharmaceutical compound;
the nanoparticles formed in-situ inside pores in the ice.

10. A piece of ice as claimed in claim 9, wherein
the piece of ice is formed in a well in a 24-well microplate;
the well providing the confined surface.

11. A piece of ice as claimed in claim 9, wherein
the piece of ice is laid over with a frame that is removable from the ice;
the frame providing the confinement of a surface of the piece of ice.

12. A piece of ice as claimed in claim 9, wherein
the piece of ice comprises at least one depression;
the depression having a base and surrounding walls,
the surrounding walls providing the confinement of the base of the depression,
wherein the base of the depression provide said surface of the piece of ice.
